(19) United States
(12) Patent Application Publication
(10) Pub. No.: US 2007/0012322 A1
(43) Pub. Date: Jan. 18, 2007

(54) Method and device for determining a position at which to deliver a substance

(75) Inventor: Wolfram Ragg, Baar (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: Disetronic Licensing AG, a Swiss corporation

(21) Appl. No.: 11/440,373

(22) Filed: May 24, 2006

Related U.S. Application Data
(63) Continuation of application No. PCT/EP04/10201, filed on Sep. 13, 2004.

(30) Foreign Application Priority Data
Nov. 28, 2003 (DE) 103 55 673.7
Feb. 13, 2004 (DE) 10 2004 007 169.1

(51) Int. Cl. A61B 19/00

(52) U.S. Cl. 128/898

(57) ABSTRACT

A method for determining a position or location for delivering a substance including providing at least one piece of reference information or a reference point, determining a location for the delivery based on the piece of reference information or in relation to the provided reference point, and outputting the determined location. The invention encompasses a device for use in the method, the device including a memory which stores reference data or patient-specific data, a computer which determines a position based on the data stored in the memory, and an output unit which outputs the determined position.

### CROSS-REFERENCE TO RELATED APPLICATION(S)

This application is a continuation of International Application No. PCT/EP2004/010201, filed on Sept. 13, 2004, which claims priority to German Application No. 103 55 673.7, filed on Nov. 28, 2003 and German Application No. 10 2004 007 169.1, filed on Feb. 13, 2004, the entire contents of all of which are incorporated herein by reference.

### BACKGROUND

The present invention relates to devices for dispensing, injecting or delivering substances, and to methods of making and using such devices. More particularly, the present invention relates to a method and a device for determining a position in order to carry out the delivery or discharge of a substance. In some embodiments, it relates to determining a position for an infusion site where, for example, an infusion pump discharges a substance into a body via a needle or cannula.

The external administration of substances through the skin, for example insulin for diabetes therapy, and into subcutaneous fatty tissue plays an important role in the treatment of diseases or metabolic disturbances. The type of appliance used, for example a pump for continuous infusion of a substance, and the selected infusion site in the body both play a role in respect of the efficacy of the administered substance and, in particular, in respect of the long-term success of, for example, infusion therapy. Also, to avoid infections, skin irritation or fatty tissue proliferation (lipohypertrophy) an administration or infusion site should be regularly and systematically changed.

The repositioning of an infusion device or cannula is generally done intuitively by users, based on their experience. Connective tissue may form at an infusion site, the result of which is that a user experiences less pain at the site when inserting a needle, which means that a user may tend to use the site preferentially for the infusion. However, formation of connective tissue at a repeatedly used infusion site can also lead to poorer uptake of an administered substance, for example insulin.

### SUMMARY

In one embodiment, the present invention comprises a method and a device for determining a position for carrying out the discharge or delivery of a substance, for example an infusion site.

In one embodiment, the present invention comprises a method for determining a position or location for carrying out a delivery of a substance comprising providing at least one piece of reference information or a reference point, determining a position or location for the delivery based on the piece of reference information or in relation to the provided reference point, and outputting the determined position or location.

In one embodiment, the present inventon comprises a device for use in the method, the device comprising a memory which stores reference data or patient-specific data, a computer which determines a position or location based on the data stored in the memory, and an output unit which outputs the determined position or location.

In one embodiment, the present invention relates to a method for determining a position in order to carry out substance discharge, for example for determining the position of an infusion site, in which method at least one item of reference information or a reference site is predefined. The reference information can, for example, include previous infusion sites, the efficacy of a substance administered at these sites, or information concerning a user, for example state of health, the location of scars, the body-mass index (BMI), and/or other suitable information. The reference site determined can, for example, be an anatomically characteristic point, for example the navel, a birthmark, or the position of a bone.

According to one embodiment of the present invention, an algorithm, for example one which employs at least one item of reference information, or a predefined pattern is used as a basis for determining a site, for example inside a defined area and/or relative to the reference site, at which an infusion needle (for example) is intended to be placed, after which the determined position or site is output. The reference site may also be a current infusion site, in which case the new position can be output, for example, by distance and/or angle data relative to the current infusion site. An algorithm can be used which, based on a time-dependent computing rule for example, determines new infusion positions in such a way that the position is altered in each case after a predefined constant or changing period of time, for example every 2 to 3 days. Moreover, an algorithm can also be used which has all or some of the already used infusion sites available as data and, based on the previously used infusion sites, determines a new infusion site, for example within one or more predefined areas. Likewise, the algorithm can employ the used type of infusion set in order to calculate a new infusion site. Moreover, it is possible for the algorithm to determine the new infusion site based on the type of substance to be administered. Particularly when rapid-action insulin and sustained-action insulin are to be administered simultaneously or one after the other, the position of the new infusion site can be determined such that the latter is favorable for the type of substance that is to be administered. For example, rapid-action insulin is preferably administered in the area of the abdomen, whereas sustained-action insulin is preferably administered in the area of the thigh.

An image of one or more administration areas is advantageously recorded, for example a photograph of the abdomen area, hip, buttocks, thigh, calf or upper arm, and is used to present the position of a new infusion site. Distinctive anatomical features of a user, for example scars or birthmarks, are indicated on such a photograph and can serve as a means of orientation in finding a new infusion site.

In some embodiments, a zoom function for enlarging the image of a certain area may be used. In presenting an imaged area, it can also be advantageous to permit mirroring and/or turning of the image in order to simplify the presentation for a user and to avoid confusion.

The administration area can, for example, be divided up into zones in order to assist in the orientation of a user or, for example, to predefine an area in which a proposed new infusion site or location, determined according to the invention, lies.

In some embodiments, certain zones that as far as possible are not to be used for the infusion are determined or predefined, for example the navel, the tissue immediately surrounding the navel, the waist, any other area where clothing would rub against an infusion needle or an infusion catheter or would limit the infusion site, scars, or areas of lipohypertrophy.

In some embodiments, a print-out can be generated, for example on a film that can be placed on a corresponding zone of the body so as to provide a printed map for the routing of the infusion sites.

In one embodiment of the method according to the present invention, the time, that is to say the day, hour and minute, when the infusion site was last changed is recorded, and this is used as a basis for determining the time when the current infusion site ought to be changed again. In some embodiments, on expiration of a predefined period of time, or on expiration of a period of time determined by an algorithm, a reminder is output, for example an optical, acoustic, sensory or other type of signal, intended to remind the user to change the current infusion site.

In some embodiments, a measurement of the action or efficacy of the administered substance is carried out, for example a measurement of the blood glucose level in the case of insulin administration, to determine from this information the efficacy of the substance or medicament. From this, it is possible to determine, for example, a local or general absorption factor, by which it is possible to establish at which infusion site the action of an administered substance was somewhat better or somewhat poorer. These values can be used when determining new positions, particularly in a user-specific manner, to select those ones that have proven especially suitable in respect of a certain substance.

In some embodiments, the algorithm used according to the invention employs further parameters or data, for example, patient-specific parameters that may possibly have an influence on the efficacy of the administered substance, for example the user's state of health, the existence of stress or strains, the performance of sports activities, the intake of meals or medicaments or the consumption of alcohol, or situations that are not everyday occurrences and that may affect the normal rhythm of life.

Based on the above-mentioned parameters and/or on other of the abovementioned features, for example the history of the previously used infusion sites or the duration of the infusion carried out at the infusion sites, it is possible to determine which areas are well suited for an infusion, for example because a good uptake or action of the administered substance has been determined at these areas. In some embodiments, the method according to the invention or the algorithm may employ or provide a learning function, which is based on a neuronal network, to determine, from the previously collated data, a new position for administration of a substance.

In some preferred embodiments, with the method according to the invention, positions for administration of a substance can be determined for several persons (so-called client capability). Thus, for example, a physician or a nurse can use a single device for carrying out the method according to the invention to control or monitor the conduct of an infusion therapy course extending over a fairly long period of time and involving a plurality of persons. In this connection, the method according to the present inventon may be used for several patients simultaneously, or patterns, trends or common features can be transferred to several patients within a certain type.

According to a further aspect of the present invention, a computer program which, when loaded in a computer or microporcessor or running on a computer, executes or is able to execute a method such as described herein. The present invention encompasses a program storage medium or computer program product with such a program.

In one embodiment, the present invention comprises a device for determining a position to carry out substance discharge, for example an infusion position, with a memory in which it is possible to store one or more of the above-described parameters or features that can be used to determine such a position. The device according to the invention also comprises a computing unit that is able to execute an algorithm with which a new position for carrying out substance discharge can be calculated or determined.

In one embodiment, the device according to the present invention also has an output unit, for example an interface for output to a display instrument or a screen on which the position determined by the computing unit can be output and, for example, presented in graph form. In some preferred embodiments, the device according to the present invention is intended to be configured such that one or more steps of the above-described method can be carried out with the device. The device according to the invention can, for example, be embodied as what is called a mobile device, e.g., a PDA, or as a stationary device using a PC. It is likewise possible to integrate the device into an infusion pump.

In some embodiments, the device according to the present invention has an input unit which can be used to input parameters that may be relevant for determining a new position, for example the type of infusion set used, the nature of the substance to be administered, or parameters that influence or characterize the current physical stress and/or state of health of a user.

In some embodiments, the device according to the present invention has a scanner or a camera which can read in or photograph an area of relevance to the positioning of, for example, an infusion needle or cannula. In some embodiments, the abdomen and hip area, thighs or arms can be recorded, and the resulting images of the respective areas can be used for presenting a new infusion position. It is possible, for example, to use image-processing algorithms to mark the areas in which no infusion is to be carried out.

In some embodiments, the device according to the present invention comprises a time-recording device or clock in order, on one hand, to record the duration of the infusion carried out at a certain site, and, on the other hand, to output, upon expiration of a predetermined or calculated time interval, a reminder that the infusion site is to be changed again.

In some embodiments, the device according to the invention is connected to at least one sensor with which an action of the substance administered at an infusion site can be measured. One exemplary sensor is a glucose sensor for carrying out a blood glucose measurement. Such sensors are known in the prior art and need not be described any further here. In some embodiments, the device has an interface for data transmission, for example a radio interface or infrared interface, to transmit data to an external appliance and/or to receive data from an external appliance, e.g., a glucose sensor.

The invention also relates to a system comprising a device as described above, and an infusion device, for example a pump, a needle or a cannula, that can be coupled to the device according to the present invention.

### BRIEF DESCRIPTION OF THE DRAWINGS

FIG. 1 shows a body with possible infusion areas schematically indicated;

FIG. 2 shows an embodiment of an input menu of a device for mobile diabetes data management (DDM);

FIG. 3 shows an example of an abdomen image with different infusion sites;

FIG. 4 shows an information window merged into the image shown in FIG. 3;

FIG. 5 shows a possible subdivision of an infusion area in quadrants.

### DETAILED DESCRIPTION

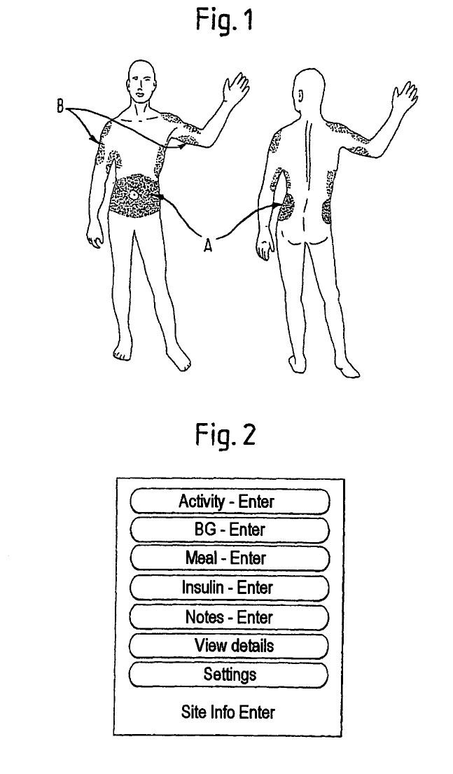

FIG. 1 shows an example of possible infusion areas A and B within which individual infusion sites are intended to be determined by the method and/or the device according to the present invention. Particularly preferred areas A are shaded dark in FIG. 1 and include the area of the abdomen, with the exception of the navel, and the hips. Alternative infusion areas B are shaded lighter in FIG. 1 and lie in the region of the shoulders, upper arms and thighs.

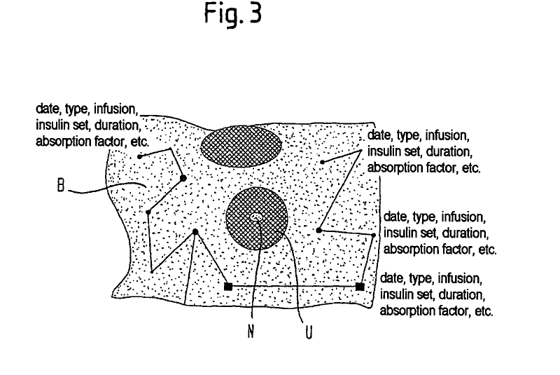

FIG. 2 shows an embodiment of an input menu which can be output from a mobile diabetes data management (DDM) system in the form of a PDA. It is possible to input details on whether the user is undertaking a sports activity or is subject to some other form of physical strain. A measured blood glucose value can also be input. It is likewise possible to input details on whether the user has just taken a meal.

Details can also be input on whether insulin has been administered to the user and, if so, when and how much. Further observations can be input by a user if he or she considers certain circumstances to be possibly relevant to conducting an infusion treatment.

A device according to one embodiment of the present invention, which provides the interface shown in FIG. 2 for interaction with a user, can be used by users of insulin pumps which are intended to reposition an infusion set within two to three days depending on the model of infusion set used and on the state of a used infusion site. Frequent and regular changing of the infusion site avoids the occurrence of infections and scars and reduces the risk of occlusions, which reduce or even completely interrupt the delivery of insulin. In order to avoid this, the present invention comprses determining a new infusion site or position which is 1 cm or more away from the last used infusion site.

According to one embodiment of the present invention, therefore, an infusion site manager is provided with which the change of site of an infusion set can be predefined and monitored or recorded to obtain data or information with which the infusion therapy can be improved or optimized. For this purpose, according to the invention, a specific area shown in FIG. 1 is selected and a new infusion site is proposed within this area, it being possible for the previously used infusion site to lie in the same area or in another area. This is done taking into account the coordinates of zones which are not suitable for an infusion, for example the navel and the tissue surrounding the latter. A new infusion site is determined using data that have already been defined and recorded, for example data that can be input using the menu shown in FIG. 2, or data that has been predefined by a physician. The determination and calculation of a new infusion site can be based on the amount of insulin that is to be administered or has already been administered, on recorded pain levels experienced by a user, and on infections that have occurred and have been recorded at certain sites, to name but a few citeria. Any suitable data or information may be used.

In accordance with an embodiment of the present invention, the aim of an algorithm for locating a new infusion site in accordance with the present invention is to create a process or method for finding, within a permitted area, the best possible position where there will probably be few side effects and where the action of the administered substance can be optimized as far as possible, as a result of which it may be possible for a smaller concentration or amount of an active substance to be administered.

According to one embodiment of the present invention, the sensitivity of a certain site to the administration of a substance can be determined, so that it is possible to establish, for example, whether a certain substance is to be administered preferably in the area of the abdomen or in the area of the thigh.

In some embodiments, it is also possible to determine sites at which the efficacy of a substance was low or at which side effects, for example increased connective tissue formation or scar formation, have occurred, so that these areas will, as far as possible, not be proposed or used for further administration of the substance.

In some embodiments, it is also possible to store certain parameters of the infusion device, e.g., the type of needle used, a needle/cannula angle, the needle/cannula length, or the material from which the needle or cannula is made, or the number of needles in a “multiple needle patch.” Thus, it can be determined whether a certain type of cannula is more suitable at specific sites than another type of cannula, and, based on this intelligence, and with determination of a new infusion position, it is also possible to determine which type of infusion device should be used at the new infusion site.

FIG. 3 shows an example of an area of the abdominal wall B. No infusion is to be carried out at the navel N itself or in the environment U of the navel. Located above the navel N in FIG. 3, there is another area not suitable for the infusion, since in this area, for example, a user has too much connective tissue.

Solid circles in FIG. 3 indicate sites at which an infusion has been carried out with a Teflon needle, the size of the circle being proportional to the duration of the administration of insulin. Solid rectangles indicate sites at which an infusion has been carried out with a steel cannula, and the chronological sequence of the infusion is identified by a line connecting the individual infusion sites. According to the present invention, starting from a current infusion site, a determination is made as to where a possibly favorable new infusion site may lie. For each infusion site, it is advantageous to record when this infusion site was used, which type of infusion set was employed, how long the infusion was carried out at this site, and what absorption factor was determined for the administered substance. It is also possible to record other parameters that could possibly be of relevance in determining the efficacy of an infusion.

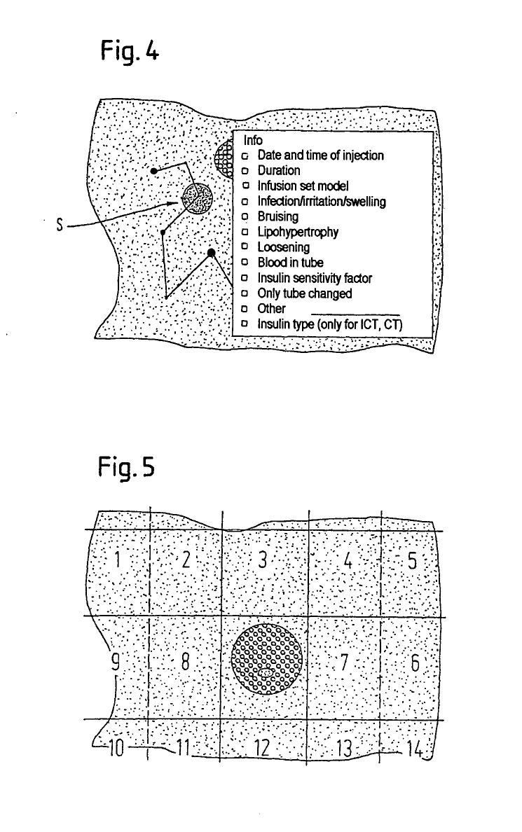

FIG. 4 shows an information window which is generated for a site S marked by a large circle and into which it is possible to enter various parameters which are or were possibly characteristic in the conduct of the infusion at this site, for example information concerning infections, irritation, swelling, bruising, fat hypertrophy, loosening of the infusion device, blood passing from the body into the infusion device, or other parameters.

FIG. 5 shows an example of how the image of an abdominal area shown in FIG. 3 can be subdivided into quadrants, which makes it easier for a user to locate a certain site, or which can be used to process data recorded during the infusion.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A method for determining a site at which an infusion of a substance can take place, said method comprising the steps of:
predefining at least one of one item of reference information or a reference site,
determining the site based on said reference information or relative to the reference site, and
outputting the determined site.

2. The method as claimed in claim 1, wherein the site is determined using an algorithm that employs at least one of predefined body-specific data, predefined infusion-specific data, infusion position patterns and data recorded during an infusion.

3. The method as claimed in claim 2, wherein the locations of previously used infusion sites are recorded and employed by the algorithm.

4. The method as claimed in claim 2, further comprising employing data relating to the type of infusion set used.

5. The method as claimed in claim 2, further comprising employing information relating to the type of substance to be infused.

6. The method as claimed in claim 2, further comprising using an image of one or more infusion areas to determine or to represent the location of a determined site.

7. The method as claimed in claim 2, wherein a predefined infusion area is divided into zones.

8. The method as claimed in claim 7, wherein in the predefined infusion area regions are defined in which no infusion is to be carried out.

9. The method as claimed in claim 6, wherein a representation of a recorded infusion area can be at least one of enlarged and mirrored.

10. The method as claimed in claim 9, wherein a printout with one of more of the determined sites and the recorded infusion areas is created.

11. The method as claimed in claim 2, wherein the time during which an infusion is carried out at a site is recorded.

12. The method as claimed in claim 1, further comprising determining a new site after a predefined or determined period of time.

13. The method as claimed in claim 1, in which the efficacy of the infused substance is measured and used in the determination of a new site.

14. The method as claimed in claim 1, in which patient-specific parameters are used in determining the site.

15. The method as claimed in claim 1, wherein the method is used on different users.

16. A computer program which, when loaded in a computer or running on a computer, executes a method comprising the steps of predefining at least one of one item of reference information or a reference site, determining the site based on said reference information or relative to the reference site, and outputting the determined site.

17. A program storage medium or computer program product with the computer program as defined in claim 16.

18. A device for determining a position to carry out a substance discharge, said device comprising a memory for storing items of reference information or patient-specific parameters, a computing unit for determining the position based on the data stored in the memory unit, and an output unit that outputs the determined position.

19. The device as claimed in claim 18, further comprising an input unit for input of patient-specific or therapy-specific parameters.

20. The device as claimed in claim 18, further comprising a camera or a scanner for recording at least one injection area.

21. The device as claimed in claim 18, further comprising a time-recording device.

22. The device as claimed in claim 18, further comprising a sensor for measuring the efficacy of the discharged substance.

23. The device as claimed in claim 18, further comprising a display device for displaying the determined position.

24. The device as claimed in claim 18, further comprising a printer for printing out an infusion schedule.

25. The device as claimed in claim 24, wherein said printing is done on a transparent film.

26. A system comprising an infusion device and a device for determining a position to carry out an infusion, said device for determining comprising a memory for storing items of reference information or patient-specific parameters, a computing unit for determining the position based on the data stored in the memory unit, and an output unit that outputs the determined position.
